(19) United States
(12) Patent Application Publication
(10) Pub. No.: US 2004/0029910 A1
(43) Pub. Date: Feb. 12, 2004

(54) Protoberberine derivatives which inhibit activity of the mitogen-activated protein kinase

(76) Inventors: Ki-Won Song, Seoul (KR); Mi-Ri Jwa, Seoul (KR); Myoung-Jin Jang, Seoul (KR); Young-Ki Paik, Seoul (KR); Jun-Ho Kim, Seoul (KR)

Correspondence Address: FOLEY AND LARDNER, SUITE 500, 3000 K STREET NW, WASHINGTON, DC, 20007, US

(21) Appl. No.: 10/311,094

(22) PCT Filed: Jun. 14, 2001
(86) PCT No.: PCT/KR01/01025

(30) Foreign Application Priority Data
Jun. 15, 2000 (KR) 2000/0033047

(51) Int. Cl. A61K 31/473

(52) U.S. Cl. 514/298

(57) ABSTRACT

The present invention relates to protoberberine derivatives which inhibit the activity of mitogen-activated protein kinase. More specifically, the present invention is directed to protoberberine derivatives which inhibit the activity of Wis1 and Spc1 kinase in intracellular signal transduction cascade wherein mitogen-activated protein kinase (MAPK) is involved.

### TECHNICAL FIELD

The present invention relates to protoberberine derivatives which inhibit the activity of mitogen-activated protein kinase. More specifically, the present invention is directed to protoberberine derivatives which inhibit the activity of Wis1 and Spc1 kinase in the intracellular mitogen-activated protein kinase(MAPK) signal transduction cascade.

### BACKGROUND ART

The intracellular signal transduction system regulates cell growth and differentiation crucial for maintenance of life. Particularly, signal transduction systems transduce extracellular stimuli including growth factors, cytokines or hormones to the cell through kinase cascade in which activated upstream kinase by mitogens in turn activate downstream kinases.

The signal transduction system transduce extracellular signals ultimately to the nucleus through intracellular intermediate messengers and thereby activate transcription regulatory factors and other target proteins, which leads to cell growth and differentiation. This mechanism is maintained in various organisms ranging from yeasts to mammalian cells [Davis, R. J., et al., TIBS, 19, 470 (1994)].

It has been known that there are three types of mitogen-activated protein kinase signaling pathways that recognize environmental change and then transmit the signal into the cell, thereby maintainig cell shape in the fission yeast Schizosaccharomyces pombe. In S. pombe, various mutants in mitosis and signal transduction are accumulated and molecular genetic approach is available.

The signal is transmitted through the pathways wherein mitogen-activated protein kinase kinase kinase(MAPKKK), mitogen-activated protein kinase kinase (MAPKK) and mitogen-activated protein kinase(MAPK) are activated sequentially [Nemin, A., TIGS, 9, 390(1993) Marshall, C. J., Cell, 80, 179(1995)].

There is a ras signaling pathway by which cells responds to nutrient exhaustion in media and to mating pheromone and thus transmit the signal to the cytosol, thereby inducing cell shape change. Another pathway is a mitogen-activated protein kinase signaling pathway by which extracellular stress including osmotic shock is transmitted to the cytosol employing Wis1 and Spc1 kinase. The other is Spm1 mitogen-activated protein kinase signaling pathway which responds to extracellular stimuli such as osmotic shock, and thereby regulates cell wall regeneration and cytokinesis.

The malfunction of genes in mitogen-activated protein kinase singal transduction pathways has no effect on cell growth in normal condition. However, the change in environmental conditions cause inhibition of cell growth, excess mitosis, or cell shape change, since cell can not respond to environmental changes.

Protoberberine derivatives of the present invention are synthesized from berberine compound, and exhibit lethal or growth inhibition activity against fungi such as Aspergillus or Candida including cutaneous filamentous fungi. Protoberberine derivatives act on the transition step of lanosterol to ergosterol by 7-reduction enzyme, exhibiting antifungal activity by inhibiting enzyme involved in the synthesis of chitin, major component of the cell wall, as well as having effect on formation of the cell membrane. However, the mechanism by which these antifungal agents inhibit physiological activity, has not yet been known.

The object of the present invention is to provide the usage of protoberberine derivatives as an inhibitor of specific mitogen-activated protein kinase(MAPK), more specifically, an inhibitor of specific Wis1 and Spc1 kinase in the MAPK singal transduction pathway in Saccharomyces pombe, since the mechanisms by which environmental changes are recognized, extracellular stimuli are transmitted to the cell, as well as the cell shape is maintained, are well understood in S. pombe

### DISCLOSURE OF INVENTION

Protoberberine derivatives of the present invention have the following chemical formula(I)

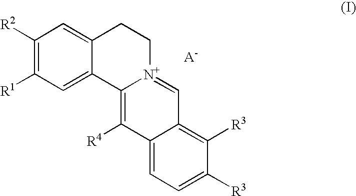

wherein R¹, R² and R³ may be the same or different, and represent C₁-C₅ alkoxy, R⁴ represents hydrogen or a group having the following chemical formula(II) and A⁻ represents inorganic acid ion, organic acid ion or

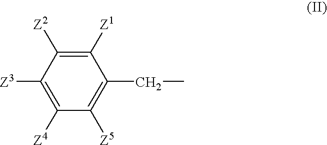

halide

wherein Z¹, Z², Z³, Z⁴ and Z⁵ may be the same or different, independently of one another represent hydrogen, halogen, C₁-C₅ alkyl, trifluoromethyl, phenyl, substituted phenyl, nitro, C₁-C₄ alkoxy, trifluoromethoxy, hydroxy, t-butyl dimethylsilyloxy, phenoxy, vinyl or methoxycarboxyl groups.

Among the compounds having the above chemical formula(I), the compound wherein R¹ and R² are methylenedioxy(-O—CH₂—O—), R³ is methoxy, and R⁴ is 3,4-dimethylbenzyl; the compound wherein R¹ and R² are methylenedioxy(-O—CH₂—O—), R³ is methoxy, and R⁴ is 4-(t-butyl)benzyl; the compound wherein R¹, R² and R³ are methoxy, and R⁴ is 4-isopropylbenzyl; the compound wherein R¹ and R² are methylenedioxy(-O—CH₂—O—), R³ is methoxy, and R⁴ is 4-(t-butyldimethylsilyloxy)benzyl; the compound wherein R¹, R² and R³ are methoxy, and R⁴ is 4-(t-butyl dimethylsilyloxy)benzyl; the compound wherein R¹, R² and R³ are methoxy, and R⁴ is 4-phenylbenzyl; or the compound wherein R¹, R² and R³ are propoxy, and R⁴ is hydrogen, are more desirable for the inhibitor of mitogen-activated protein kinase in the present invention.

Protoberberine derivatives which inhibit mitogen-activated protein kinase of the present invention, 13-benzylberberine iodide, 13-(4-chlorobenzyl)berberine iodide, 13-(3-bromobenzyl)berberine iodide, 13-(2,3-dichlorobenzyl)berberine chloride, 13-(2-chloro-6-fluoro benzyl)berberine chloride, 13-(4-fluoro-2-trifluoro methylbenzyl)berberine chloride, 13-(2,3,4,5,6-pentafluoro benzyl)berberine chloride, 13-(2,3,5,6-tetrafluoro-4-tri fluoromethylbenzyl)berberine chloride, 13-(4-methylbenzyl) berberine chloride, 13-(2,4-dimethylbenzyl)berberine chloride, 13-(4-methyl-3-nitrobenzyl)berberine chloride, 13-(trifluoromethylbenzyl)berberine chloride, 13-(4-methoxybenzyl)berberine chloride, 13-(4-trifluoromethoxy benzyl)berberine chloride, 13-(4-methoxycarbonylbenzyl) berberine chloride, 13-(4-t-butylbenzyl)berberine chloride, 13-(4-i-propylbenzyl)berberine chloride, 13-(4-(t-butyl dimethylsilyloxy)benzyl)berberine chloride, 13-(4-t-butylbenzyl)palmatine iodide, 13-(4-(t-butyldimethyl silyloxy)benzyl)palmatine chloride, 2,3,9,10-tetrapropoxy protoberberine iodide, which is described in detail in the specification of the Korean patent application No. 258,849 and U.S. Pat. No. 6,008,356.

In the present invention, Shizosaccharomyces pombe was cultured and minimum concentrations of protoberberines for its inhibition, was determined. Then, the knock-out mutants in three MAP kinase cascades described above were treated with protoberberine derivatives and their effects on the growth of each mutant were measured. The results show that protoberberine derivatives have an effect on knock-out mutant of wis1 and spc1. Also, it was studied whether protoberberine derivatives act on whole transduction pathway wherein Wis1 MAPKK and Spc1 MAPK are involved or only on Wis1 MAPKK and Spc1 MAPK. From the result that protoberberine derivatives do not act on knock-out mutants of the genes for MAPKKK that function upstream of wis1 and spc1, win1 and wis4, it was found that these protoberberine derivatives selectively inhibit the activity of Wis1 and Spc1 kinase.

### BRIEF DESCRIPTION OF THE DRAWINGS

FIG. 1 illustrates three signal transduction pathways wherein mitogen-activated protein kinases are involved in Schizosaccharomyces pombe in the present invention.

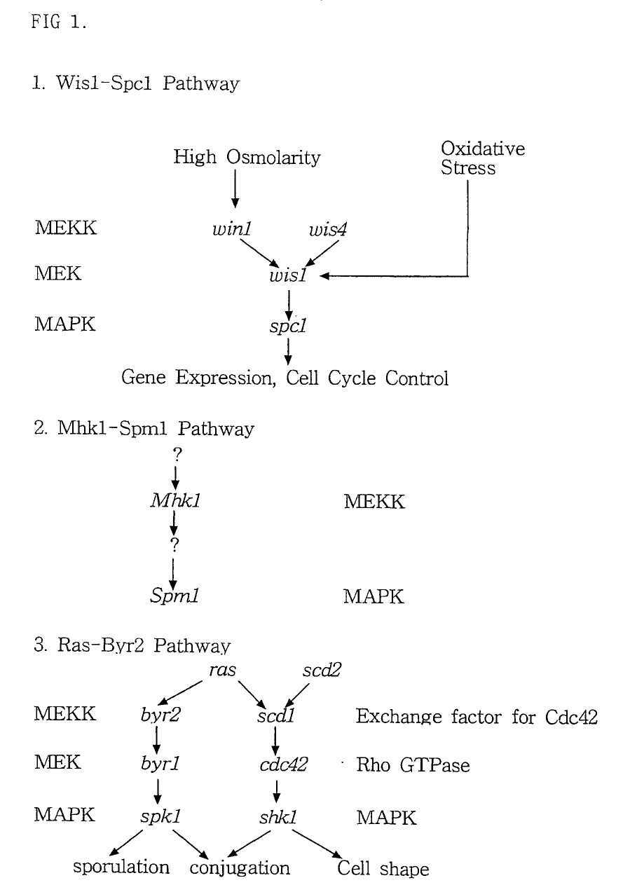

FIG. 2 represents the photograph of electroporesis showing inhibition of the activity of Wis1 kinase by protoberberine derivatives of the present invention.

FIG. 3 represents the photograph of electroporesis showing inhibition of the activity of Spc1 kinase by protoberberine derivatives of the present invention.

### BEST MODE FOR CARRYING OUT THE INVENTION

The usage of protoberberine derivatives as the inhibitor of MAPK in the present invention will be in more detail illustrated by the following examples and attached figures. But it should be understood that the present invention is not limited to these examples in any manner.

### EXAMPLE 1

Measurement of Minimum Inhibitory Concentration(MIC) of Protoberberine Derivatives on Schizosaccharomyces pombe

KGY246 cells, wild type Saccharomyces pombe, were cultured in rich media YE^{AU} (yeast extract, peptone, malt extract, dextrose, agar: pH 6.2)containing 0.75 mg/L adenine and uracil to exponential phase. To determine minimum inhibitory concentration of each protoproberberine derivative, cells in exponential phase were transferred to YE^{AU} medium containing variable concentrations of protoberberine derivatives and cell number was measured every 3 hour. Thereafter, the minimal concentration on which cell number was maintained constantly was determined as minimum inhibitory concentration. The minimum inhibitory concentrations of protoberberine derivatives 13-(4-t-butylbenzyl)berberine chloride, 13-(4-(t-butyldimetylsilyloxy)benzyl)berberine chloride and 2,3,9,10-tetrapropoxyprotoberberine iodide of the present invention were 20 ug/mL, 7 ug/mL and 20 ug/mL respectively. To minimize inhibition by solvent itself, 10% ethanol was used as a solvent of protoberberine derivatives. The processes of cell culture and measurement of cell number described above were repeated using 13-benzylberberine iodide, 13-(4-chlorobenzyl)berberine iodide, 13-(3-bromobenzyl)berberine iodide, 13-(2,3-dichlorobenzyl) berberine chloride, 13-(2-chloro-6-fluorobenzyl)berberine chloride, 13-(4-fluoro-2-trifluoromethylbenzyl)berberine chloride, 13-(2,3,4,5,6-pentafluorobenzyl)berberine chloride, 13-(2,3,5,6-tetrafluoro-4-trifluoromethylbenzyl) berberine chloride, 13-(4-methylbenzyl)berberine chloride, 13-(2,4-dimethylbenzyl)berberine chloride, 13-(4-methyl-3-nitrobenzyl)berberine chloride, 13-(3-trifluoromethyl benzyl)berberine chloride, 13-(4-methoxybenzyl)berberine chloride, 13-(4-trifluoromethoxybenzyl)berberine chloride, 13-(4-methoxycarbonylbenzyl)berberine chloride, 13-(4-i-propylbenzyl)berberine chloride. These compounds also had the minimum concentration of inhibition at the concentration of 18-22 ug/mL.

### EXAMPLE 2

Effects of Protoberberine Derivatives on Various Knock-Out Mutants of Schizosaccharomyces pombe

To investigate the effect of protoberberine derivatives on the growth of cells, the cells of each strain were cultured in rich medium YE^{AU} to exponential phase(5×10⁷ cells/mL). These cells were transferred to YE^{AU} medium and cultured for 12 hours. Then cell number was measured every 3 hours. Specificity of protoberberine derivatives on cell growth was proved through comparison with the result obtained by treatment with 3 ug/mL miconazole of which inhibiting effect of yeast cell growth has been known.

The strains of Schizosaccharomyces pombe used in the present invention are indicated in table 1.

TABLE 1

| strain | Genotype | Characteristic |
|---|---|---|
| KGY246 | h⁻leu1-32ura4-D18ade6 | Wild type |
| KS1366 | h⁻leu1-32Δ spc1 | spc1 knock-out |
| JM544 | h⁻leu1-32Δ wis1 | wis1 knock-out |
| ED1292 | h⁻leu1-32ura4-D18wis4::ura4+ | wis4 knock-out |
| ED1388 | h⁻leu1-32ura4-D18ade6-M216win1::ura4+ | win1 knock-out |
| TZs69 | h⁻leu1-32ura4-D18Δ spm1 | spm1 knock-out |
| SPSU | h⁹⁰ leu1-32ura4-D18ade6Δ byr2 | byr2 knock-out |

The growth of wild type Schizosaccharomyces pombe was inhibited by protoberberine derivatives as indicated in table 2. The knock-out mutants in the MAPK pathways were treated with protoberberine derivatives of the present invention and the results were indicated in tables in below. As indicated in table 5, the knock-out mutant of wis1 encoding mitogen-activated protein kinase kinase(MAPKK) was not inhibited by 13-(4-t-butylbenzyl) berberine chloride, 13-(4-(t-butyldimethylsilyloxy) benzyl) berberine chloride and 2,3,9,10-tetrapropoxyprotoberberine iodide. As indicated in table 6, the knock-out mutant of spc1 encoding Spc1 mitogen-activated protein kinase(MAPK) which is phosphorylated and thereby activated by Wis1 mitogen-activated protein kinase kinase(MAPKK) was not inhibited at all by 13-(4-(t-butyldimethylsilyloxy)benzyl) berberine chloride and partially inhibited by 13-(4-t-butylbenzyl)berberine chlroride whereas completely inhibited by 2,3,9,10-tetrapropoxy protoberberineiodide. That is, 13-(4-t-butylbenzyl)berberine chloride, 13-(4-(t-butyldimethylsilyloxy)benzyl)berberine chloride and 2,3,9,10-tetrapropoxyprotoberberine iodide inhibit the activity of Wis1 kinase and 2,3,9,10-tetrapropoxy protoberberine iodide inhibit the activity of Spc1 kinase. The effects of each compound 13-benzylberberine iodide, 13-(4-chlorobenzyl) berberine iodide, 13-(3-bromobenzyl) berberine iodide, 13-(2,3-dichlorobenzyl) berberine chloride, 13-(2-chloro-6-fluorobenzyl)berberine chloride, 13-(4-fluoro-2-trifluoro methylbenzyl)berberine chloride, 13-(2,3,4,5,6-pentafluoro benzyl)berberine chloride, 13-(2,3,5,6-tetrafluoro-4-trifluoromethylbenzyl)berberine chloride, 13-(4-methylbenzyl)berberine chloride, 13-(2,4-dimethylbenzyl) berberine chloride, 13-(4-methyl-3-nitrobenzyl)berberine chloride, 13-(trifluoromethylbenzyl)berberine chloride, 13-(4-methoxybenzyl)berberine chloride, 13-(4-trifluoro methoxybenzyl)berberine chloride, 13-(4-methoxycarbonyl benzyl)berberine chloride, 13-(4-i-propylbenzyl)berberine chloride on each knock-out mutant were measured. As a result, these compounds also inhibited the activity of Wis1 kinase.

TABLE 2
Effects of a protoberberine derivative 13-(4-(t-
butyldimethylsilyloxy)benzyl)berberine
chloride on several kinase mutants.

| | | wis1 | spc1 | Spm1 | byr2 | |
|---|---|---|---|---|---|---|
| | | knock- | knock- | knock- | knock- | |
| time | KGY246 | out | out | out | out | Ethanol |
| 14 | 20 | 257 | 57 | 55 | 34 | 427 |
| 17 | 19 | 665 | 90 | 94 | 72 | 695 |
| 20 | 22 | 1355 | 173 | 266 | 156 | 1515 |
| 23 | 16 | 3100 | 210 | 450 | 292 | 3050 |

TABLE 3
Effects of protoberberine derivatives on the knock-out
mutant of byr2 in Ras-Byr2 pathway

| | 13- | | | |
|---|---|---|---|---|
| | (4-t-butylbenzyl) | 2,3,9,10-tetrapropoxy | | |
| time | berberine chloride | protoberberine iodide | miconazole | ethanol |
| 12 | 156 | 220 | 32 | 344 |
| 15 | 150 | 207 | 19 | 735 |
| 18 | 147 | 185 | 31 | 1220 |
| 21 | 136 | 167 | 30 | 1811 |
| 24 | 128 | 155 | 27 | 3436 |

TABLE 4
Effects of protoberberine derivatives on the knock-out
mutant of spm1 in Mhk1-Spm1 pathway

| | 13- | | | |
|---|---|---|---|---|
| | (4-t-butylbenzyl) | 2,3,9,10-tetrapropoxy | | |
| Time | berberine chloride | protoberberine iodide | miconazole | ethanol |
| 12 | 89 | 117 | 28 | 107 |
| 15 | 113 | 4 | 27 | 224 |
| 18 | 131 | 7 | 23 | 305 |
| 21 | 118 | 165 | 34 | 547 |
| 24 | 125 | 228 | 26 | 1236 |

TABLE 5
Effects of protoberberine derivatives on the knock-out
mutant of wis1

| | 13- | | | |
|---|---|---|---|---|
| | (4-t-butylbenzyl) | 2,3,9,10-tetrapropoxy | | |
| time | berberine chloride | protoberberine iodide | miconazole | ethanol |
| 12 | 154 | 164 | 35 | 163 |
| 15 | 319 | 325 | 37 | 345 |
| 18 | 538 | 502 | 25 | 681 |
| 21 | 1265 | 1041 | 19 | 1357 |
| 24 | 1788 | 1574 | 12 | 2184 |

TABLE 6
Effects of protoberberine derivatives on the knock-out
mutant of spc1

| | 13- | | | |
|---|---|---|---|---|
| | (4-t-butylbenzyl) | 2,3,9,10-tetrapropoxy | | |
| Time | berberine chloride | protoberberine iodide | miconazole | ethanol |
| 12 | 156 | 201 | 15 | 158 |
| 15 | 299 | 382 | 9 | 312 |
| 18 | 488 | 676 | 12 | 634 |
| 21 | 874 | 1232 | 18 | 1258 |
| 24 | 1083 | 1634 | 17 | 1746 |

### EXAMPLE 3

Effects of Protoberberine Derivatives on the Knock-Out Mutant of win1 and wis4

The knock-out mutant of win1 and wis4 was cultured and treated with 13-(4-t-butylbenzyl)berberine chloride, 13-(4-(t-butyldimethylsilyloxy)benzyl)berberine chloride or 2,3,9,10-tetrapropoxyprotoberberine iodide of the compounds 13-benzylberberine iodide, 13-(4-chloro benzyl)berberine iodide, 13-(3-bromobenzyl)berberine iodide, 13-(2,3-dichlorobenzyl)berberine chloride, 13-(2-chloro-6-fluorobenzyl)berberine chloride, 13-(4-fluoro-2-trifluoromethylbenzyl)berberine chloride, 13-(2,3,4,5,6-pentafluorobenzyl)berberine chloride, 13-(2,3,5,6-tetra fluoro-4-trifluoromethylbenzyl)berberine chloride, 13-(4-methylbenzyl)berberine chloride, 13-(2,4-dimethylbenzyl) berberine chloride, 13-(4-methyl-3-nitrobenzyl)berberine chloride, 13-(3-trifluoromethylbenzyl)berberine chloride, 13-(4-methoxybenzyl)berberine chloride, 13-(4-trifluoro methoxybenzyl)berberine chloride, 13-(4-methoxycarbonyl benzyl)berberine chloride, 13-(4-t-butylbenzyl)berberine chloride, 13-(4-1-propylbenzyl) berberine chloride, 13-(4-(t-butyldimethylsilyloxy)benzyl) berberine chloride, 13-(4-t-butylbenzyl)palmatine iodide, 13-(4-(t-butyldimethyl silyloxy)benzyl)palmatine chloride and 2,3,9,10-tetra propoxyprotoberberine iodide. As indicated in table 7 and 8, the knock-out mutant of win1 and wis4 encoding the two upstream kinases which phosphorylate wis1 and spc1 were completely inhibited by protoberberine derivatives as in wild type, which indicates that these protoberberine derivatives have effect on specific Wis1 and Spc1 kinases and on the not whole Wis1-Spc1 mitogen-activated protein kinase signal transduction pathway.

TABLE 7
Effect of protoberberine derivatives on the knock-out
mutant of win1

| | 13-(4-t- | 2,3,9,10- | | |
|---|---|---|---|---|
| | butylbenzyl) | tetrapropoxy | 13-(4-(t- | |
| | berberine | protoberbrine | butyldimethylsilyloxy) | |
| time | chloride | iodide | benzylberberine chloride | DMSO |
| 13 | 28 | 102 | 31 | 155 |
| 16 | 26 | 210 | 16 | 355 |
| 19 | 17 | 423 | 16 | 725 |
| 22 | 37 | 750 | 26 | 1140 |
| 25 | 13 | 1575 | 33 | 2625 |

TABLE 8
Effect of protoberberine derivatives on the knock-out
mutant of wis4

| | 13-(4-t- | 2,3,9,10- | | |
|---|---|---|---|---|
| | butylbenzyl) | tetrapropoxy | 13-(4-t- | |
| | berberine | protoberberine | butyldimethylsilyloxy | |
| time | chloride | iodide | chloride | DMSO |
| 13 | 24 | 96 | 37 | 465 |
| 16 | 45 | 113 | 52 | 970 |
| 19 | 36 | 265 | 25 | 2800 |
| 22 | 23 | 645 | 36 | 4050 |
| 25 | 19 | 1250 | 23 | 8500 |

### EXAMPLE 4

Induction of Expression and Purification of Wis1 and Spc1 Kinases

The genes wis1 and spc1 were cloned into the pRep1 vector containing nmt1 promoter of which expression was inhibited by thiamin in the medium and able to tag GST. The KGY246 cells, wild type Schizosaccharomyces pombe cells were transformed with this plasmid. The expression of Wis1 and Spc1 kinase tagged with GST was induced by the cultivation of the transformed cells in MM^{AU} medium containing 0.4 mM thiamin followed by cultivation to exponential phase(5×10⁶ cells) for about 20 hours in 100 mL of MM^{AU} medium in which thiamin was withdrawn. After osmotic shock(0.6M KCl) and oxidative stress were given to the cells expressing Wis1 and Spc1 tagged with GST for 25 minutes, cells were recovered at 4° C., washed with ice-cold PBS/NaF(50 mM)/NaN₃(1 mM) buffer and HB buffer(25 mM HEPES, pH 7.5, 60 mM β-glycerophosphate, 15 mM EGTA, 0.1 mM Na vanadate, 0.1% triton and hydrolyzed using glass bead and bead beater purchased from Sigma. The supernatant of hydrolyzed cells was incubated with 100 mM NaCl for salting-out to remove unnecessary proteins. After the supernatant was incubated with 0.045 g/ml GST beads for 1.5 hours, proteins not bound to GST beads were removed. Purified Wis1 and Spc1 kinases were separated on 7% SDS-PAGE and the degree of purification was confirmed by western blotting using GST antibody.

### EXAMPLE 5

The Activity of Wis1 and Spc1 Kinase and the Effect of Protoberberine Derivatives on Kinase Activity

Before Wis1 and Spc1 kinases purified in example 4 were analyzed, they were pre-incubated in analyzing buffer of kinase (50 mM Tris-Cl pH7.4, 20 mM MgCl₂, 5 mM EGTA, 2 mM Na vanadate, 1 mM DTT). The kinase reaction was performed at 30° C. for 30 minutes in the presence of 1 mg/ml myelin basic protein(MBP), 1 mM ATP and 20 uCi of [γ-³²p]ATP and then terminated by adding SDS sample buffer and heating at 90° C. for 5 minutes. The reaction mixtures were separated on SDS-polyacrylamide gel electrophoresis. After fixation with 10% acetic acid: 10% methanol and drying, the gel was exposed on X-ray film. When Wis1 is activated by stimulus, it also activates autophosphorylation. Therefore, the degree of Wis1 autophosphorylation was also measured.

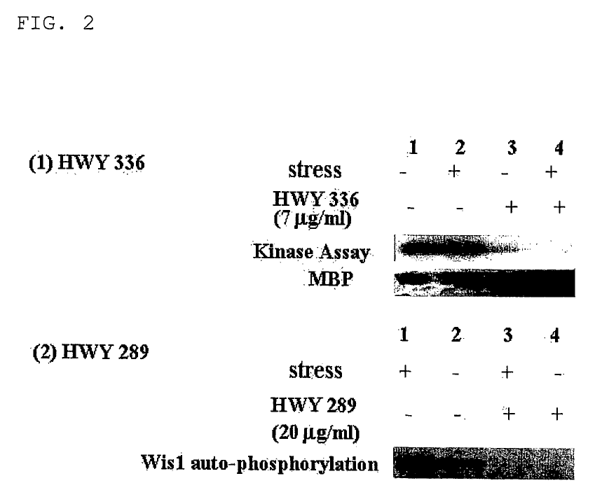

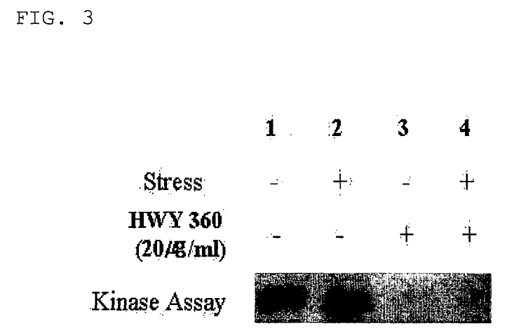

As represented in FIG. 2, Wis1, mitogen-activated protein kinase(MAPK) kinase is directly inhibited by 13-(4-(t-butyldimethylsilyloxy)benzylberberine chloride and 13-(4-t-butylbenzyl)berberine chloride. In additon, the processes of incubation, kinase reaction and measurement of the degree of effect on autophosphorylation were repeated using 13-benzylberberine iodide, 13-(4-chlorobenzyl)berberine iodide, 13-(3-bromobenzyl)berberine iodide, 13-(2,3-di chlorobenzyl)berberine chloride, 13-(2-chloro-6-fluoro benzyl)berberine chloride, 13-(4-fluoro-2-trifluoromethyl benzyl)berberine chloride, 13-(2,3,4,5,6-pentafluorobenzyl berberine chloride, 13-(2,3,5,6-tetrafluoro-4-trifluoro methylbenzyl)berberine chloride, 13-(4-methylbenzyl) berberine chloride, 13-(2,4-dimethylbenzyl)berberine chloride, 13-(4-methyl-3-nitrobenzyl)berberine chloride, 13-(3-trifluoromethylbenzyl)berberine chloride, 13-(4-methoxybenzyl)berberine chloride, 13-(4-trifluoromethoxy benzyl)berberine chloride, 13-(4-methoxycarbonylbenzyl) berberine chloride, 13-(4-1-propylbenzyl)berberine chloride. The activity of Wis1 kinase was directly inhibited by these compounds. As represented in FIG. 3, the Spc1, mitogen-activated protein kinase, was only directly inhibited by 2,3,9,10-tetrapropoxy protoberberine iodide when myelin basic protein was used as a substrate.

Industrial Applicability

The present invention provides protoberberine derivatives which inhibit specifically the activity of Wis1 and Spc1 kinase in the mitogen-activated protein kinase signal transduction pathway, and protoberberine derivatives of the present invention can be used as a reagent for the study of intracellular signal transduction pathways. Also, it can be used for the development of pharmaceutical product which regulates the activity of mitogen-activated protein kinase such as an inhibitor of SAPK(stress activated protein kinase)/JNK(c-Jun N-terminal kinase) kinase or anticancer drug, since the mitogen-activated protein kinase signal transduction system in Schizosaccharomyces pombe is highly homologous to the SAPK/JNK signaling pathway which transmits environmental stimuli such as cytokines or DNA damages in mammalian cells.

What is claimed is:

1. An inhibitor for mitogen-activated protein kinase, of protoberberine derivative having the following chemical formula(I) 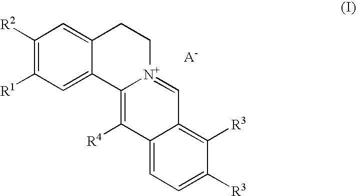
wherein R¹, R² and R³ may be the same or different, and represent C₁-C₅ alkoxy, R⁴ represents hydrogen or a group having the following chemical formula(II), and A⁻ represents inorganic acid ion, organic acid ion or halide: 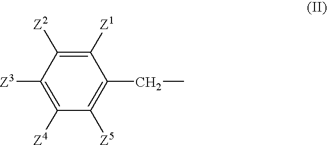
wherein Z¹, Z², Z³, Z⁴ and Z⁵ may be the same or different, independently of one another represent hydrogen, halogen, C₁-C₅ alkyl, trifluoromethyl, phenyl, substituted phenyl, nitro, C₁-C₄ alkoxy, trifluoromethoxy, hydroxy, t-butyldimethylsilyloxy, phenoxy, vinyl or methoxycarboxyl groups.

2. The inhibitor for mitogen-activated protein kinase, protoberberine derivative according to claim 1, wherein R¹-R² is methylenedioxy(-O—CH₂—O—), R³ is methoxy, R⁴ is 4-(t-butyl)benzyl and A⁻ is chloride.

3. The inhibitor for mitogen-activated protein kinase, of protoberberine derivative according to claim 1, wherein R¹-R² is methylenedioxy(-O—CH₂—O—), R³ is methoxy, R⁴ is 4-(t-butyldimethylsilyloxy)benzyl and A⁻ is chloride.

4. The inhibitor for mitogen-activated protein kinase, of protoberberine derivative according to claim 1, wherein R¹-R³ is propoxy, R⁴ is hydrogen and A⁻ is iodide.
